(19) United States
(12) Patent Application Publication
(10) Pub. No.: US 2012/0179274 A1
(43) Pub. Date: Jul. 12, 2012

(54) BIFURCATED, MULTI-PURPOSE PROSTHETIC FOOT

(76) Inventor: Roland J. Christensen, Gunnison, UT (US)

(21) Appl. No.: 13/179,145

(22) Filed: Jul. 8, 2011

Related U.S. Application Data
(60) Provisional application No. 61/363,679, filed on Jul. 13, 2010.

(51) Int. Cl. A61F 2/66

(52) U.S. Cl. 623/55

(57) ABSTRACT

A prosthetic foot has a plurality of curvilinear leaf springs coupled to an attachment member, including a primary curvilinear leaf spring forming a forefoot arc, an elongated curvilinear leaf spring disposed below the forefoot arc, and a secondary curvilinear leaf spring forming a secondary forefoot member. The forefoot arc is laterally bifurcated while the forefoot reinforcement arc is laterally unitary.

### PRIORITY CLAIM

Priority is claimed to copending U.S. Provisional Patent Application Ser. No. 61/363,679, filed Jul. 13, 2010, which is hereby incorporated herein by reference in its entirety.

### BACKGROUND

1. Field of the Invention

The present invention relates generally to a prosthetic foot.

2. Related Art

High performance carbon fiber foot prosthetics have been developed to replace an amputee's foot. For example, see U.S. Pat. Nos. 4,547,913; 6,805,717; 6,911,052; 6,929,665; and 7,419,509. Such feet often include a flexible forefoot and heel. Sometimes the forefoot is split to simulate toe rotation and/or accommodate sloped terrain. For example, see U.S. Pat. Nos. 4,645,509; 5,181,933; 5,514,185; 5,776,205; and 6,071,313. It has been proposed to include a secondary foot member that is engaged during extreme use or greater force. For example, see U.S. Pat. Nos. 5,944,760 and 6,241,776. The development of prosthetic feet is an ongoing endeavor.

### SUMMARY OF THE INVENTION

It has been recognized that it would be advantageous to develop a bifurcated leaf spring foot to provide toe rotation and multipurpose use for normal and extreme activity. In addition, it has been recognized that it would be advantageous to develop a leaf spring foot that increases stiffness with increased activity level or extra weight.

The invention provides a prosthetic foot with an attachment member configured to be coupled to a stump of an amputee. The foot also has a pair of curvilinear leaf springs with attachment ends coupled together at the attachment member and emanating therefrom to form vertically oriented and forwardly concave oriented arcs. The arcs include: 1) a primary forefoot spring arc terminating in a toe end at a toe location of a natural foot, and 2) a secondary forefoot reinforcement spring arc terminating in a free end elevated above a portion of the primary forefoot spring arc so that the arcs are vertically bifurcated. The arcs have the same shape, with the secondary forefoot reinforcement spring arc smaller than and nesting within a concave space of the primary forefoot spring arc. The primary forefoot spring arc is laterally bifurcated from a toe end to an intermediate location intermediate the toe end and the attachment end, but at least along a majority of a longitudinal length thereof. The foot also has a footplate coupled to the primary forefoot spring arc and extending from the toe end to a heel end at a heel location of the natural foot. The footplate is longitudinally split along a longitudinal length thereof The secondary forefoot reinforcement spring arc is laterally unitary along a longitudinal length thereof, and has a width laterally spanning a gap between opposite lateral sides of the primary forefoot spring arc, and a majority of opposite lateral sides of the primary forefoot spring arc along essentially the longitudinal length of the secondary forefoot reinforcement spring arc and a majority of a longitudinal length of the gap.

In addition, the invention provides a prosthetic foot with an attachment member configured to be coupled to a stump of an amputee. The foot also has a pair of curvilinear leaf springs with attachment ends coupled together at the attachment member and emanating therefrom to form vertically oriented and forwardly concave oriented arcs. The arcs include: i) a primary forefoot spring arc terminating in a toe end at a toe location of a natural foot, and ii) a secondary forefoot reinforcement spring arc terminating in a free end elevated above a portion of the primary forefoot spring arc so that the arcs are vertically bifurcated. The arcs have the same shape with the secondary forefoot reinforcement spring arc smaller than and nesting within a concave space of the primary forefoot spring arc. The primary forefoot spring arc is laterally unitary along substantially an entire longitudinal length thereof A footplate is coupled to the primary forefoot spring arc and extends from the toe end to a heel end at a heel location of the natural foot. The footplate is laterally unitary along substantially an entire longitudinal length thereof. The secondary forefoot reinforcement spring arc is laterally unitary along a longitudinal length thereof and has a width laterally spanning a majority of the primary forefoot spring arc along essentially the longitudinal length of the secondary forefoot reinforcement spring arc.

In addition, the invention provides a prosthetic foot with an attachment member configured to be coupled to a stump of an amputee. The foot also has a curvilinear leaf spring with an attachment end coupled at the attachment member and emanating therefrom to form a vertically oriented and forwardly concave oriented primary forefoot spring arc terminating in a toe end at a toe location of a natural foot. The primary forefoot spring arc is laterally bifurcated from a toe end to an intermediate location intermediate the toe end and the attachment end, but at least along a majority of a longitudinal length thereof. A secondary forefoot reinforcement spring arc is coupled to the primary forefoot spring arc and emanates from the toe end of the primary forefoot spring arc and terminates in a free end elevated above an arch portion of the primary forefoot spring arc so that the arcs are vertically bifurcated. The secondary forefoot reinforcement spring arc is at least laterally bifurcated from the toe end. A footplate is coupled to the primary forefoot spring arc and extends from the toe end to a heel end at a heel location of the natural foot. The footplate is longitudinally split along a longitudinal length thereof.

Furthermore, the invention provides a prosthetic foot with an attachment member configured to be coupled to a stump of an amputee. The foot also has a curvilinear leaf spring with an attachment end coupled at the attachment member and emanating therefrom to form a vertically oriented and forwardly concave oriented primary forefoot spring arc terminating in a toe end at a toe location of a natural foot. The primary forefoot spring arc is laterally unitary along substantially an entire longitudinal length thereof. A secondary forefoot reinforcement spring arc is coupled to the primary forefoot spring arc and emanates from the toe end of the primary forefoot spring arc and terminates in a free end elevated above an arch portion of the primary forefoot spring arc so that the arcs are vertically bifurcated. The secondary forefoot reinforcement spring arc is laterally unitary along substantially an entire longitudinal length thereof A footplate is coupled to the primary forefoot spring arc and extends from the toe end to a heel end at a heel location of the natural foot. The footplate is laterally unitary along substantially an entire longitudinal length thereof

### BRIEF DESCRIPTION OF THE DRAWINGS

Additional features and advantages of the invention will be apparent from the detailed description which follows, taken in conjunction with the accompanying drawings, which together illustrate, by way of example, features of the invention; and, wherein:

FIG. 1a is a perspective view of a prosthetic foot in accordance with an embodiment of the present invention;

FIG. 1b is a side view of the prosthetic foot of FIG. 1a;

FIG. 1c is a cross-sectional side view of the prosthetic foot of FIG. 1a;

FIG. 2 is a side view of another prosthetic foot in accordance with another embodiment of the present invention;

FIG. 3 is a side view of another prosthetic foot in accordance with another embodiment of the present invention;

FIG. 4 is a perspective view of another prosthetic foot in accordance with another embodiment of the present invention;

FIG. 5a is a side view of another prosthetic foot in accordance with another embodiment of the present invention;

FIG. 5b is a cross-sectional side view of the prosthetic foot of FIG. 5a;

FIG. 6 is a cross-sectional side view of another prosthetic foot in accordance with another embodiment of the present invention;

FIG. 7 is a side view of another prosthetic foot in accordance with another embodiment of the present invention;

FIG. 8a is a side view of another prosthetic foot in accordance with another embodiment of the present invention;

FIG. 8b is a side view of the prosthetic foot of FIG. 8a; and

FIG. 9 is a cross-sectional side view of another prosthetic foot in accordance with another embodiment of the present invention.

Reference will now be made to the exemplary embodiments illustrated, and specific language will be used herein to describe the same. It will nevertheless be understood that no limitation of the scope of the invention is thereby intended.

### DETAILED DESCRIPTION OF EXAMPLE EMBODIMENT(S)

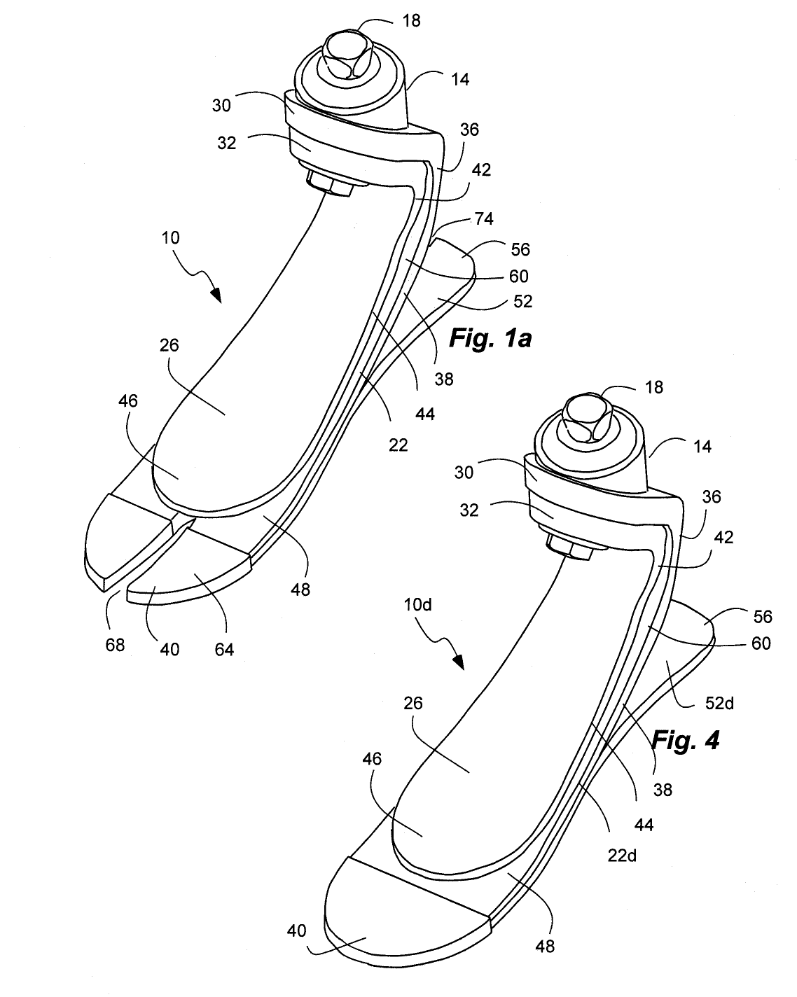

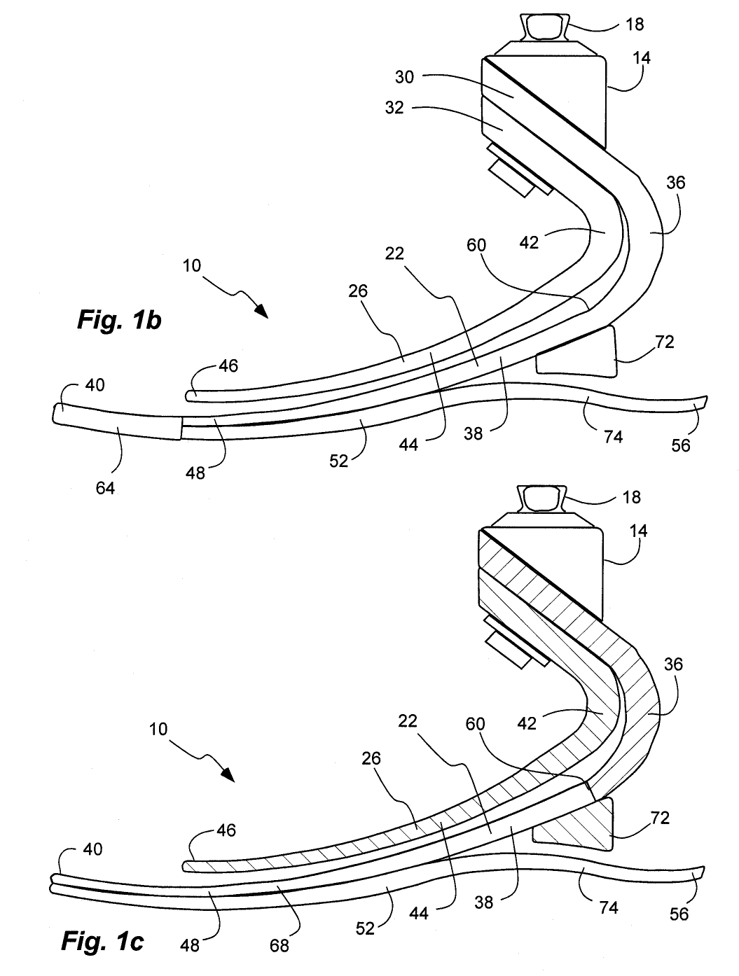

As illustrated in FIGS. 1a-1c, a prosthetic foot, indicated generally at 10, in an example implementation in accordance with the invention is shown. The prosthetic foot 10 can be coupled to a limb of an amputee, such as with a socket that fits over the limb. The prosthetic foot can include an attachment member 14 with a standard attachment fixture 18 that is received in a corresponding fixture on a pylon or socket, as is known in the art. The foot 10 can be received in a cavity of a shell shaped as a natural foot, such as a polyurethane foam; or can be received with in a standard shoe; or both. Alternatively, the foot can be configured for use on the ground without any intervening shell or shoe. Thus, the foot contacts a support surface, which can be the inside top surface of the shell or an insole of the shoe, or can be the ground.

The foot 10 can include a plurality of leaf springs that can bend and deflect under force to store energy, and can return to their original shape releasing energy as the force is removed. Thus, as the amputee walks on the foot, the leaf springs store and return energy. The leaf springs can be flexible and resilient, and can be composite, including a fiber, such as graphite, in a resin matrix. The springs can be straight or flat in the lateral, or side to side, direction, while being curved in the longitudinal, or front to back, direction, and curved in the vertical, or top to bottom, direction.

The foot 10 includes a pair of curvilinear leaf springs, including a primary curvilinear leaf spring or primary forefoot spring arc 22 defining a primary forefoot member, and a secondary curvilinear leaf spring or secondary forefoot reinforcement spring arc 26 defining a secondary forefoot member. The primary foot member can be primarily used during normal activities, such as walking, while the secondary forefoot member can be engaged in extreme activities, such as running or lifting. The primary leaf spring or forefoot arc 22 has an attachment end 30 coupled or affixed to the attachment member 14. Similarly, the secondary leaf spring or forefoot reinforcement arc 26 has an attachment end 32 coupled to the attachment end 30 of the primary leaf spring 22 and attachment member 14. Thus, the attachment ends 30 and 32 can abut to one another, can border one another or be contiguous with one another, and can be coupled together at the attachment member, and the arcs can emanate therefrom. The curvilinear leaf springs can form vertically oriented and forwardly concave oriented arcs. The arcs can be generally curved or overall curved, without necessarily having the same curvature or axis of rotation along the length thereof, and may be all curved or curved along the length thereof, or simply include curved parts or portions separated by straight segments. The forefoot arc 22 and the forefoot reinforcement arc 26 can be curved in a vertically oriented plane aligned with a longitudinal axis (forward and rearward) with the foot. In addition, the forefoot arc and the forefoot reinforcement arc 26 can be concave with the concave arc facing forwardly.

The primary leaf spring or forefoot arc 22 can extend: i) downward and rearward through the attachment end 30, ii) downward and curved through and ankle section 36, iii) downward and forward through an arch section 38, and iv) forward to a toe end 40 at a toe location of a natural foot. Thus, the forefoot arc emanates from the attachment end 30 and terminates at the toe end 40. Similarly, the secondary leaf spring or forefoot reinforcement arc 26 can extend: i) downward and rearward through the attachment end 32, ii) downward and curved through and ankle section 42, iii) downward and forward through an arch section 44, and iv) forward to a free end 46. Thus, the forefoot reinforcement arc emanates from the attachment end 30 and terminates at the free end 46.

The free end 46 can be elevated above a portion of the primary forefoot spring arc. The free end 46 of the secondary forefoot reinforcement spring arc 26 can terminate longitudinally behind the toe end 40 of the primary forefoot spring arc 22 and above a ball location 48 of the natural foot. Thus, the arcs 22 and 26 are vertically bifurcated (or split into two at the toe and free ends from a lateral perspective with the free end elevated higher than the toe end). (The forefoot arc and the forefoot reinforcement arc can be vertically bifurcated from the toe end beyond the intermediate location 60.) In addition, the arcs 22 and 26 can have substantially or essentially the same shape, with the secondary forefoot reinforcement spring arc 26 being smaller than and nesting within a concave space of the primary forefoot spring arc 22. Thus, the arcs 22 and 26 are concentric in the sense that one arc is within another, but non-concentric in that they do not share a common effective axis or center, but instead share a common point along their curvature or perimeter, namely the attachment ends. Alternatively, the arcs 22 and 26 can have different shapes. The bifurcated or separated ends 40 and 46 of the arcs 22 and 26 allow the foot to provide a more universal use, with the primary arc 22 providing a majority of the support during a majority of activities, such as walking and standing, while the secondary arc 26 is engaged during more extreme activities, such as running or lifting. The secondary arc 26 or forefoot reinforcement arc increases the stiffness of the foot with increased activity level or weight increase, such as carrying a weight. Thus, the foot provides multiple uses without the amputee having to exchange prosthetics or springs for different activities during a day. The forefoot arc 22 deflects towards the forefoot reinforcement arc 26, and towards the attachment end or member, on toe off during gait. Similarly, during extreme activity, the forefoot arc 22 contacts and engages the forefoot reinforcement arc 26 and both deflect towards the attachment end or member.

The foot 10 can also include another curvilinear leaf spring, namely an elongated curvilinear leaf spring disposed below the forefoot arc 22 and affixed to the toe end 40 of the forefoot arc and extending therefrom to form a footplate 52 terminating in a heel end 56 at a heel location of the natural foot. Thus, the footplate 52 can extend a longitudinal length of the foot. The footplate 52 can be essentially flat and horizontal, but can have vertical curvatures including a downward curvature or valley at the ball and heel locations, and upward curvature or peaks at the toe and arch locations (at rearmost end). The footplate 52 can abut to the forefoot arc 22 from the toe end, through the ball section, and into the arch section. The forefoot arc 22 and the footplate 52 can be vertically bifurcated from the heel end 56 of the footplate and the ankle 36 of the forefoot arc. (The forefoot arc and the footplate can be vertically bifurcated from the heel end beyond the intermediate location 60.) Thus, the heel end 56 of the footplate can deflect towards the forefoot arc on heel strike during gait.

In addition to the various leaf springs being vertically bifurcated, one or more of the leaf springs can also be laterally bifurcated. The primary forefoot spring arc or forefoot arc 22 can be laterally bifurcated from the toe end 40 to an intermediate location 60 intermediate the toe end 40 and the attachment end 32, but at least along a majority of a longitudinal length thereof. The intermediate location 60 or termination of the bifurcation or split can be disposed vertically below the attachment member 14. Thus, the forefoot arc 22 is split at the toe location 40, ball location 48 and arch location 38; while being laterally unitary or monolithic through the attachment section 32 and ankle section 36. The forefoot arc 22 is laterally bifurcated from the toe end 40 to the intermediate location 60. The bifurcation of the forefoot arc 22 allows the toe end 40 to flex independently and more like a natural foot and can allow the foot to respond to uneven terrain. Similarly, the footplate 52 can be longitudinally split along a longitudinal length thereof. Again, the split forefoot allows the toe end 40 to flex independently and more like a natural foot, and can allow the foot to respond to uneven terrain. A split attachment 64 couples corresponding opposite lateral sides of the footplate 52 and the primary forefoot spring arc 22. The attachment 64 can be formed by wrapping the toe ends of the footplate and the forefoot arc with fiber in resin matrix.

The secondary forefoot reinforcement spring arc 26 can be laterally unitary or monolithic along a longitudinal length thereof The forefoot reinforcement arc 26 can have a width laterally spanning a gap 68 between opposite lateral sides of the primary forefoot spring arc. In addition, the forefoot reinforcement arc 26 can span a majority of opposite lateral sides of the primary forefoot spring arc along essentially the longitudinal length of the secondary forefoot reinforcement spring arc, and a majority of a longitudinal length of the gap. The laterally unitary forefoot reinforcement arc 26 spanning the gap allows the opposite sides of the forefoot arc 22 to engage the forefoot reinforcement arc 26 either together or separately.

The free end 46 of the secondary forefoot reinforcement spring arc 26 terminates longitudinally behind the toe end 40 of the primary forefoot spring arc 22 and above the ball location 48 of the natural foot. In addition, the free end 46 of the secondary forefoot reinforcement spring arc 46 and the toe end 40 of the primary forefoot spring arc 22 have a same lateral curvature in an essentially horizontal plane (i.e. the curved ends when looking down upon the ends). The shape and configuration of the ends of the curvilinear spring arcs can facilitate insertion into a shell or shoe.

The foot can also include a heel reinforcement member disposed between the heel end 56 of the footplate 52 and the primary forefoot spring arc 22, or ankle portion thereof. The heel reinforcement member can be laterally unitary or monolithic, and can have a width laterally spanning a gap 74 between opposite lateral sides of the footplate, and over opposite lateral sides of the footplate. The laterally unitary heel reinforcement member spanning the gap allows the opposite sides of the footplate to engage the heel reinforcement member either together or separately. The heel reinforcement member can be a compressible block 72, such as a polyurethane block. The block 72 can have upper and lower surfaces inclined to match the forefoot arc and footplate. A gap or space can be formed between the heel reinforcement member or block 72 and the heel end 56 of the footplate 52; with the block attached to the forefoot arc 22. Thus, the footplate 52 and heel reinforcement member or block 72 are vertically bifurcated at the heel end. The heel end 56 of the footplate 52 deflects towards the heel reinforcement member or block 72, and towards the attachment end or member, on heel strike during gait. Similarly, during extreme activity, the heel end 56 of the footplate 52 contacts and engages the heel reinforcement member or block 72, with the footplate 52 deflecting and the block 72 compressing. Thus, the heel reinforcement member or block 72 increases stiffness of the foot with increased activity level or weight increase, such as carrying a load. Alternatively, a gap or space can be formed between the heel reinforcement member or block 72 and the forefoot arc 22; with the block 72 attached to the footplate 52. Thus, the forefoot arc 22 and the heel reinforcement member 72 can be vertically bifurcated.

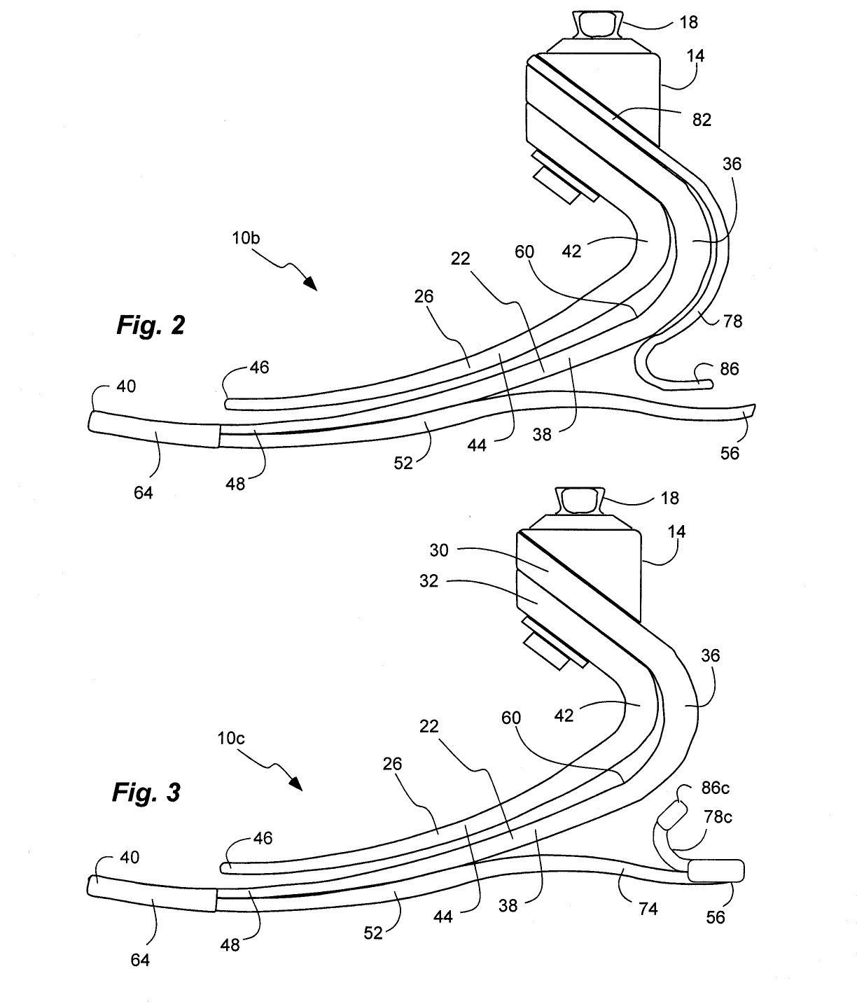

Alternatively, referring to FIG. 2, a prosthetic foot 10b similar to that described above is show, and which description is herein incorporated by reference, but the heel reinforcement member can be an elongated curvilinear heel spring 78 with an attachment end 82 coupled to the attachment member 14 and extending to a free end 86 elevated above a portion of the footplate 52 at the heel end 56. Thus, a gap is formed between the free end and the heel end. The heel spring 78 can be laterally unitary along a longitudinal length thereof, and can have a width laterally spanning the gap 74 between opposite lateral sides of the footplate and a majority of opposite lateral sides of the footplate.

A variable stiffness material can be disposed between the vertically bifurcated spring members to vary the rate of stiffening. The material can be a compressible material or a bladder or the like. The material can be disposed between the forefoot arc 22 and the forefoot reinforcement arc 26, such as at the free end. Similarly, the material can be disposed between the heel end of the footplate 52 and the heel reinforcement member 72. Similarly, the material can be disposed between the heel reinforcement member 72 and the forefoot arc 22. Thus, the springs remain vertically bifurcated, but the gap or space can be filed, wholly or partially, with a compressible material to vary the transfer of load or force from the primary spring to the secondary spring or reinforcement.

Referring to FIG. 3, a prosthetic foot 10c similar to that described above is show, and which description is herein incorporated by reference, but the heel reinforcement member can be a heel spring 78c with an attachment end coupled to the heel end 56 of the footplate 52 and extending to a free end 86c elevated above a portion of the footplate at the heel end. The attachment end of the heel spring 78c can be laterally bifurcated to match the split heel end 56 of the footplate 52. Alternatively, the heel spring can be laterally split along its entire length.

Alternatively, the heel end 56 of the footplate 52 described in the above embodiments can be laterally unitary while the toe end of the footplate is bifurcated. Referring to FIG. 4, a prosthetic foot 10d similar to that described above is show, and which description is herein incorporated by reference, but the primary curvilinear leaf spring or primary forefoot spring arc 22d and the footplate 52d can be non-bifurcated or non-split, respectively, and instead can be laterally unitary or monolithic along a longitudinal length thereof. The secondary forefoot reinforcement spring arc 26 can be laterally unitary along a longitudinal length thereof and can have a width laterally spanning a majority of the primary forefoot spring arc 22d along essentially the longitudinal length of the secondary forefoot reinforcement spring arc. Similarly, heel reinforcement member can be laterally unitary along a longitudinal length thereof and having a width laterally spanning a majority of the footplate.

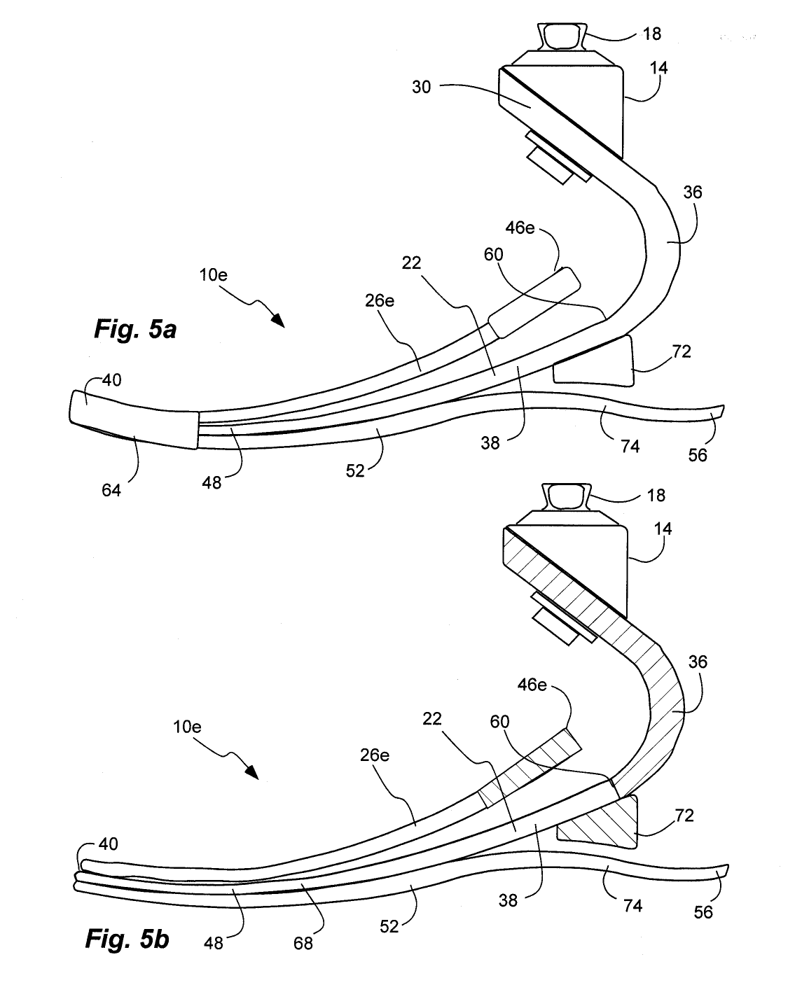

Referring to FIGS. 5a and 5b, another embodiment of a prosthetic foot 10e is shown that is similar in many respect to those described above, and which description is herein incorporated by reference. A secondary forefoot reinforcement spring arc or forefoot reinforcement arc 26e forms a secondary forefoot member coupled to the primary forefoot spring arc 22 and emanating from the toe end 40 of the primary forefoot spring arc, and terminating in a free end 46e elevated above an arch portion 38 of the primary forefoot spring arc 22 so that the arcs 22 and 26e are vertically bifurcated. Thus, the arcs 22 and 26e are separated by a gap or space at the free end 46e of the forefoot reinforcement arc 26e and the arch portion 38 of the forefoot arc 22. The bifurcated or separated arcs 22 and 26e allow the foot to provide a more universal use, with the primary arc 22 providing a majority of the support during a majority of activities, such as walking and standing, while the secondary arc 26e is engaged during more extreme activities, such as running or lifting. The secondary arc 26e or forefoot reinforcement arc increases the stiffness of the foot with increased activity level or weight increase, such as carrying a weight. Thus, the foot provides multiple uses without the amputee having to exchange prosthetics or springs for different activities during a day. The forefoot arc 22 deflects towards the attachment end or member, on toe off during gait. During extreme activity, forefoot reinforcement arc 26e deflects towards the forefoot arc 22, or arch portion 38 thereof, and contacts and engages the forefoot arc 22, and both deflect towards the attachment end or member. A variable stiffness material or bumper can be disposed between the free end 46e of the forefoot reinforcement arc 26e and the forefoot arc 22.

The secondary forefoot reinforcement spring arc 26e can be laterally unitary at the free end 46e thereof, with a width laterally spanning a gap between opposite lateral sides of the primary forefoot spring arc 22 and a majority of opposite lateral sides of the primary forefoot spring arc 22. The laterally unitary free end 46e of the forefoot reinforcement arc 26e spanning the gap allows the opposite sides of the forefoot arc 22 to engage the forefoot reinforcement arc 26e either together or separately. In addition, the free end 46e of the forefoot reinforcement arc 26e can be wrapped together with fiber in a resin matrix. The attachment end of the forefoot reinforcement arc 26e can be laterally bifurcated and attached to the laterally bifurcated toe end 40 of the forefoot arc 22. The lateral bifurcation or gap of the primary forefoot arc 22 can be longer than the lateral bifurcation or gap of the forefoot reinforcement arc 26e. In addition, the lateral bifurcation or gap of the primary forefoot arc 22 can be longer than the forefoot reinforcement arc 26e.

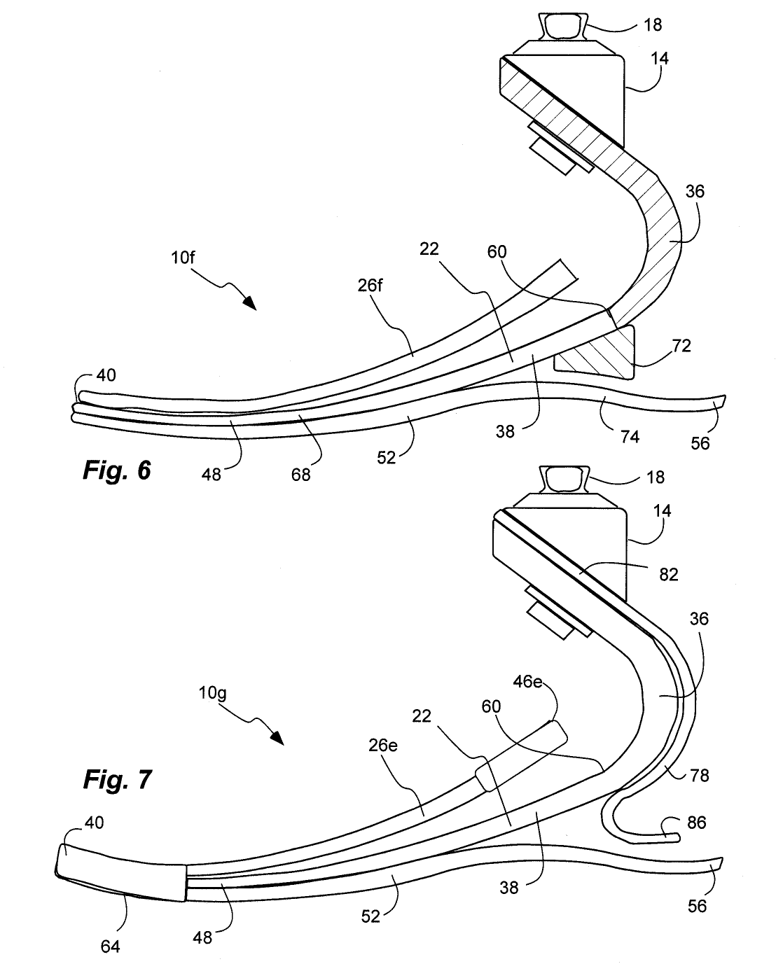

Referring to FIG. 6, another embodiment of a prosthetic foot 10f is shown that is similar in many respect to those described above, and which description is herein incorporated by reference, but with the secondary forefoot reinforcement spring arc 26f longitudinally split along a longitudinal length thereof. The split forefoot reinforcement arc 26f allows the opposite sides of the forefoot arc 22 to deflect separately and independently of one another.

Referring to FIG. 7, another embodiment of a prosthetic foot 10g is shown that is similar in many respect to those described above, and which description is herein incorporated by reference, but with the heel reinforcement member being an elongated curvilinear heel spring 78g. The curvilinear heel spring 78g can have an attachment end 82 coupled to the attachment member 14 and extending to a free end 86 elevated above a portion of the footplate 52 at the heel end 56. Thus, a gap is formed between the free end and the heel end. The heel spring 78 can be laterally unitary along a longitudinal length thereof, and can have a width laterally spanning the gap 74 between opposite lateral sides of the footplate and a majority of opposite lateral sides of the footplate.

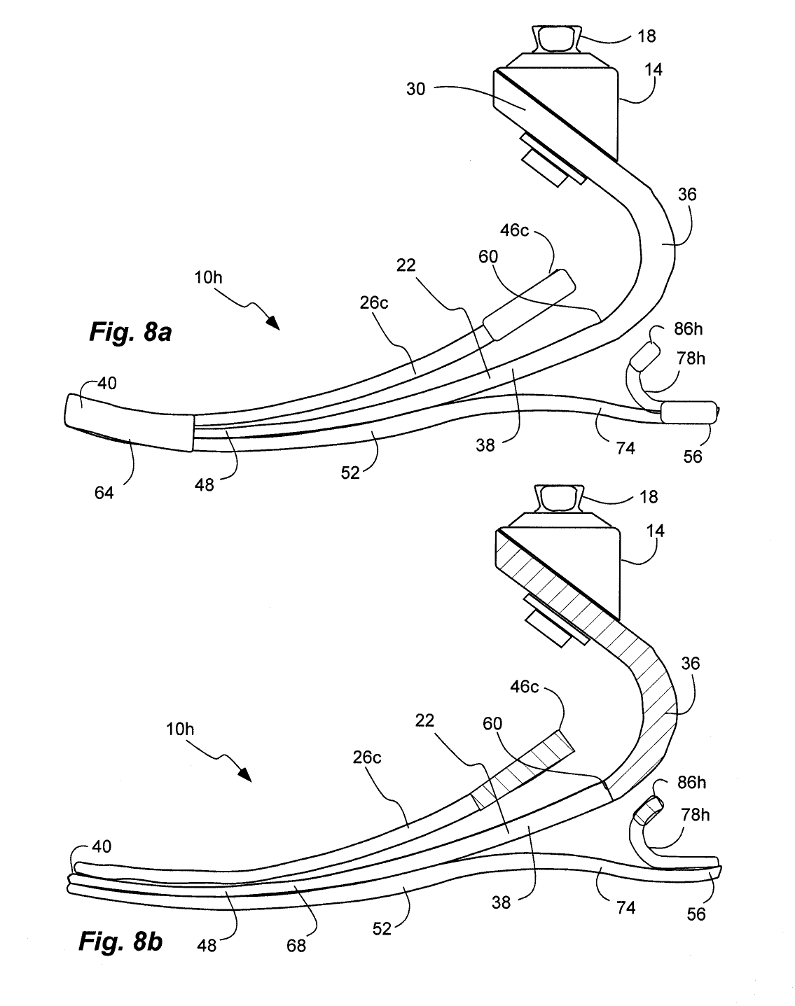

Referring to FIGS. 8a and 8b, another embodiment of a prosthetic foot 10h is shown that is similar in many respect to those described above, and which description is herein incorporated by reference, but with a heel reinforcement member such as a heel spring 78h an attachment end coupled to the heel end 56 of the footplate 52 and extending to a free end 86h elevated above a portion of the footplate at the heel end. The attachment end of the heel spring 78c can be laterally bifurcated to match the split heel end 56 of the footplate 52.

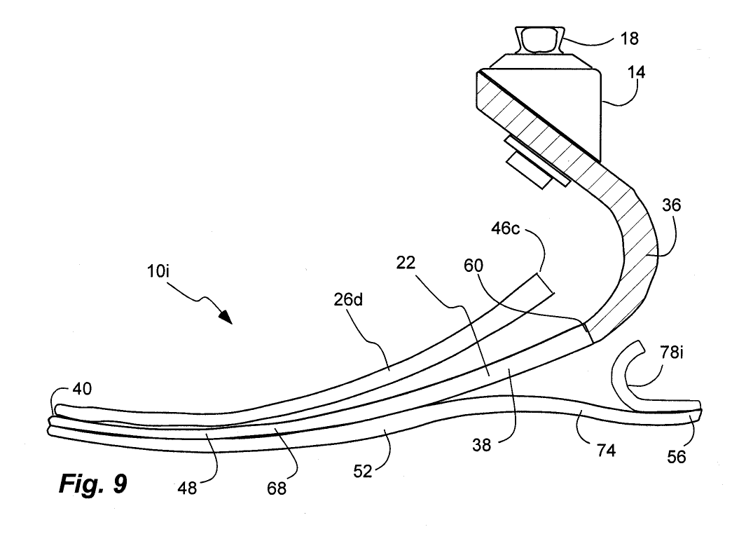

Alternatively, the heel reinforcement member 78i can be longitudinally split along a longitudinal length thereof, as shown with the prosthetic foot 10i in FIG. 9. The split heel reinforcement member 78i allows the opposite sides of the footplate 52 to deflect separately and independently of one another. The foot 10i shown in FIG. 9 is similar to those described above, and which description is herein incorporated by reference.

In addition, the prosthetic feet described above can be non-split, and can be provided with a primary forefoot spring arc and footplate that are laterally unitary along substantially an entire longitudinal length thereof. The secondary forefoot reinforcement spring arc can be laterally unitary along a longitudinal length thereof and can have a width laterally spanning a majority of the primary forefoot spring arc along essentially the longitudinal length of the secondary forefoot reinforcement spring arc. Similarly, the heel spring can be laterally unitary along a longitudinal length thereof and having a width laterally spanning a majority of the footplate.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

1. A prosthetic foot device, comprising:
a) an attachment member configured to be coupled to a stump of an amputee;
b) a pair of curvilinear leaf springs with attachment ends coupled together at the attachment member and emanating therefrom to form vertically oriented and forwardly concave oriented arcs, including: i) a primary forefoot spring arc terminating in a toe end at a toe location of a natural foot, and ii) a secondary forefoot reinforcement spring arc terminating in a free end elevated above a portion of the primary forefoot spring arc so that the arcs are vertically bifurcated, with the arcs having the same shape with the secondary forefoot reinforcement spring arc smaller than and nesting within a concave space of the primary forefoot spring arc;
c) the primary forefoot spring arc being laterally bifurcated from a toe end to an intermediate location intermediate the toe end and the attachment end but at least along a majority of a longitudinal length thereof;
d) a footplate coupled to the primary forefoot spring arc and extending from the toe end to a heel end at a heel location of the natural foot, the footplate being longitudinally split along a longitudinal length thereof; and
e) the secondary forefoot reinforcement spring arc being laterally unitary along a longitudinal length thereof and having a width laterally spanning a gap between opposite lateral sides of the primary forefoot spring arc and a majority of opposite lateral sides of the primary forefoot spring arc along essentially the longitudinal length of the secondary forefoot reinforcement spring arc and a majority of a longitudinal length of the gap.

2. A device in accordance with claim 1, wherein the free end of the secondary forefoot reinforcement spring arc terminates longitudinally behind the toe end of the primary forefoot spring arc above a ball location of the natural foot.

3. A device in accordance with claim 1, wherein the free end of the secondary forefoot reinforcement spring arc and the toe end of the primary forefoot spring arc have a same lateral curvature in an essentially horizontal plane.

4. A device in accordance with claim 1, further comprising:
a heel reinforcement member disposed between the heel end of the footplate and the primary forefoot spring arc.

5. A device in accordance with claim 4, wherein the heel reinforcement member is an elongated curvilinear heel spring with an attachment end coupled to the attachment member and extending to a free end elevated above a portion of the footplate at the heel end; and
wherein the heel spring is laterally unitary along a longitudinal length thereof and having a width laterally spanning a gap between opposite lateral sides of the footplate and a majority of opposite lateral sides of the footplate.

6. A device in accordance with claim 4, wherein the heel reinforcement member is a compressible block.

7. A device in accordance with claim 1, wherein the foot plate is an elongate curvilinear spring.

8. A device in accordance with claim 1, wherein:
the primary forefoot spring arc extends: i) downward and rearward through the attachment end, ii) downward and curved through and ankle section, iii) downward and forward through an arch section, and iv) forward to the toe end; and
the secondary forefoot reinforcement spring arc extends: i) downward and rearward through the attachment end, ii) downward and curved through and ankle section, iii) downward and forward through an arch section, and iv) forward to the free end.

9. A device in accordance with claim 1, wherein the foot plate is affixed to the primary forefoot spring arc at the toe end with a split attachment coupling corresponding opposite lateral sides of the foot plate and the primary forefoot spring arc.

10. A prosthetic foot device, comprising:
a) an attachment member configured to be coupled to a stump of an amputee;
b) a pair of curvilinear leaf springs with attachment ends coupled together at the attachment member and emanating therefrom to form vertically oriented and forwardly concave oriented arcs, including: i) a primary forefoot spring arc terminating in a toe end at a toe location of a natural foot, and ii) a secondary forefoot reinforcement spring arc terminating in a free end elevated above a portion of the primary forefoot spring arc so that the arcs are vertically bifurcated, with the arcs having the same shape with the secondary forefoot reinforcement spring arc smaller than and nesting within a concave space of the primary forefoot spring arc;
c) the primary forefoot spring arc being laterally unitary along substantially an entire longitudinal length thereof;
d) a footplate coupled to the primary forefoot spring arc and extending from the toe end to a heel end at a heel location of the natural foot, the footplate being laterally unitary along substantially an entire longitudinal length thereof; and
e) the secondary forefoot reinforcement spring arc being laterally unitary along a longitudinal length thereof and having a width laterally spanning a majority of the primary forefoot spring arc along essentially the longitudinal length of the secondary forefoot reinforcement spring arc.

11. A device in accordance with claim 10, wherein the free end of the secondary forefoot reinforcement spring arc terminates longitudinally behind the toe end of the primary forefoot spring arc above a ball location of the natural foot.

12. A device in accordance with claim 10, wherein the free end of the secondary forefoot reinforcement spring arc and the toe end of the primary forefoot spring arc have a same lateral curvature in an essentially horizontal plane.

13. A device in accordance with claim 10, further comprising:
a heel reinforcement member disposed between the heel end of the footplate and the primary forefoot spring arc.

14. A device in accordance with claim 13, wherein the heel reinforcement member is an elongated curvilinear heel spring with an attachment end coupled to the attachment member and extending to a free end elevated above a portion of the footplate at the heel end; and wherein the heel spring is laterally unitary along a longitudinal length thereof and having a width laterally spanning a majority of the footplate.

15. A device in accordance with claim 13, wherein the heel reinforcement member is a compressible block.

16. A device in accordance with claim 10, wherein the foot plate is an elongate curvilinear spring.

17. A device in accordance with claim 10, wherein:
the primary forefoot spring arc extends: i) downward and rearward through the attachment end, ii) downward and curved through and ankle section, iii) downward and forward through an arch section, and iv) forward to the toe end; and
the secondary forefoot reinforcement spring arc extends: i) downward and rearward through the attachment end, ii) downward and curved through and ankle section, iii) downward and forward through an arch section, and iv) forward to the free end.

18. A prosthetic foot device, comprising:
a) an attachment member configured to be coupled to a stump of an amputee;
b) a curvilinear leaf spring with an attachment end coupled at the attachment member and emanating therefrom to form a vertically oriented and forwardly concave oriented primary forefoot spring arc terminating in a toe end at a toe location of a natural foot;
c) the primary forefoot spring arc being laterally bifurcated from a toe end to an intermediate location intermediate the toe end and the attachment end but at least along a majority of a longitudinal length thereof;
d) a secondary forefoot reinforcement spring arc coupled to the primary forefoot spring arc and emanating from the toe end of the primary forefoot spring arc and terminating in a free end elevated above an arch portion of the primary forefoot spring arc so that the arcs are vertically bifurcated;
e) the secondary forefoot reinforcement spring arc being at least laterally bifurcated from the toe end;
f) a footplate coupled to the primary forefoot spring arc and extending from the toe end to a heel end at a heel location of the natural foot; and
g) the footplate being longitudinally split along a longitudinal length thereof.

19. A device in accordance with claim 18, wherein the secondary forefoot reinforcement spring arc is laterally unitary at the free end thereof with a width laterally spanning a gap between opposite lateral sides of the primary forefoot spring arc and a majority of opposite lateral sides of the primary forefoot spring arc.

20. A device in accordance with claim 18, wherein the secondary forefoot reinforcement spring arc is longitudinally split along a longitudinal length thereof.

21. A device in accordance with claim 18, further comprising:
a heel reinforcement member disposed between the heel end of the footplate and the primary forefoot spring arc.

22. A device in accordance with claim 21, wherein the heel reinforcement member is an elongated curvilinear heel spring with an attachment end coupled to the attachment member and extending to a free end elevated above a portion of the footplate at the heel end; and wherein the heel spring is laterally unitary along a longitudinal length thereof and having a width laterally spanning a gap between opposite lateral sides of the footplate and a majority of opposite lateral sides of the footplate.

23. A device in accordance with claim 21, wherein the heel reinforcement member is a compressible block.

24. A device in accordance with claim 21, wherein the heel reinforcement member is an elongated curvilinear heel spring with an attachment end coupled to the heel end of the footplate and extending to a free end elevated above a portion of the footplate at the heel end.

25. A device in accordance with claim 18, wherein the foot plate is an elongate curvilinear spring.

26. A device in accordance with claim 18, wherein the primary forefoot spring arc extends: i) downward and rearward through the attachment end, ii) downward and curved through and ankle section, iii) downward and forward through an arch section, and iv) forward to the toe end.

27. A device in accordance with claim 18, wherein the foot plate is affixed to the primary forefoot spring arc at the toe end with a split attachment coupling corresponding opposite lateral sides of the foot plate and the primary forefoot spring arc.

28. A prosthetic foot device, comprising:
a) an attachment member configured to be coupled to a stump of an amputee;
b) a curvilinear leaf spring with an attachment end coupled at the attachment member and emanating therefrom to form a vertically oriented and forwardly concave oriented primary forefoot spring arc terminating in a toe end at a toe location of a natural foot;
c) the primary forefoot spring arc being laterally unitary along substantially an entire longitudinal length thereof;
d) a secondary forefoot reinforcement spring arc coupled to the primary forefoot spring arc and emanating from the toe end of the primary forefoot spring arc and terminating in a free end elevated above an arch portion of the primary forefoot spring arc so that the arcs are vertically bifurcated;
e) the secondary forefoot reinforcement spring arc being laterally unitary along substantially an entire longitudinal length thereof;
f) a footplate coupled to the primary forefoot spring arc and extending from the toe end to a heel end at a heel location of the natural foot; and
g) the footplate being laterally unitary along substantially an entire longitudinal length thereof.

29. A device in accordance with claim 28, wherein the secondary forefoot reinforcement spring arc has a width laterally spanning a majority of the primary forefoot spring arc.

30. A device in accordance with claim 28, further comprising:
a heel reinforcement member disposed between the heel end of the footplate and the primary forefoot spring arc.

31. A device in accordance with claim 30, wherein the heel reinforcement member is an elongated curvilinear heel spring with an attachment end coupled to the attachment member and extending to a free end elevated above a portion of the footplate at the heel end; and wherein the heel spring is laterally unitary along a longitudinal length thereof and having a width laterally spanning a majority of the footplate.

32. A device in accordance with claim 30, wherein the heel reinforcement member is a compressible block.

33. A device in accordance with claim 30, wherein the heel reinforcement member is an elongated curvilinear heel spring with an attachment end coupled to the heel end of the footplate and extending to a free end elevated above a portion of the footplate at the heel end.

34. A device in accordance with claim 28, wherein the foot plate is an elongate curvilinear spring.

35. A device in accordance with claim 28, wherein the primary forefoot spring arc extends: i) downward and rearward through the attachment end, ii) downward and curved through and ankle section, iii) downward and forward through an arch section, and iv) forward to the toe end.
